(19) United States
(12) Patent Application Publication
(10) Pub. No.: US 2025/0090261 A1
(43) Pub. Date: Mar. 20, 2025

(54) RESILIENT EXPANDABLE STERILIZABLE PACKAGING FOR INDIVIDUAL ITEMS

(71) Applicant: Roesler IP GmbH, Hergensweiler (DE)

(72) Inventor: Thiemo Roesler, Wangen/Neuravensburg (DE)

(21) Appl. No.: 18/883,446

(22) Filed: Sep. 12, 2024

(30) Foreign Application Priority Data
Sep. 18, 2023 (EP) 23198033.5

(51) Int. Cl. A61B 50/30; B65D 85/24

(52) U.S. Cl. CPC A61B 50/30; A61B 17/865; A61F 2/0095; B65D 25/107; B65D 85/24

(57) ABSTRACT

Resilient expandable individual packaging (1, 20) for elongated items (17), consisting of a packaging body (1, 20) open on one side with a packaging space (25, 25′) closed at the bottom, which merges into a resiliently expandable closure part (6) on its upper side, wherein in the transition region between the closure part (6) and the packaging space (25, 25′) one or more locking knobs (11, 12) are arranged, which form a resiliently expandable closure gap (13, 13′) therebetween.

### TECHNICAL FIELD

The subject matter of the invention is a resiliently expandable sterilizable packaging for packaging singular items in the medical field according to the preamble of claim 1.

### SUMMARY

This type of resiliently expandable individual packaging is used as packaging for sterile medical items, which are used in particular in surgery, orthopedics, and other medical fields. Such items must be able to be removed from their packaging during the operation-preferably with one hand-easily, without disruption, and while maintaining sterility.

The purpose of the individual packaging according to the prior art and the present invention is to provide a sterilizable packaging for elongated items of any kind, which is preferably sterilizable by radiation.

From DE 10 2013 019 452 B4, a resiliently expandable individual packaging for elongated items has become known, wherein the packaged item consists of at least one head of enlarged diameter and an adjoining bolt part of reduced diameter. The individual packaging is formed from an approximately cylindrical packaging sleeve which is open at least on one side at the front and which consists of a resiliently bendable material and which has at least one upper insertion opening for inserting or removing the elongated item, whereby only the head of the item to be held is held in a position-secured manner in the region of the insertion opening of the packaging.

A disadvantage is that the known individual packaging is dependent on holding the head of an elongated item—e.g., a screw-which severely limits the field of application. Other items that do not have such a head with an enlarged diameter cannot be held in a position-secured manner.

Another disadvantage of the known individual packaging is that the individual packaging has to be individually adapted to the diameter of the screw head, which means that a large number of individual packages have to be produced that are adapted to different screw shapes and screw lengths as well as head diameters.

There is only a small grip region, which is only present in the region of the expandable neck of the individual packaging, which means that the gripping surfaces are small and therefore difficult to operate.

Since the screw head of the screw to be held is located in the region of the insertion opening, there is a risk that an operator in the sterile surroundings of the operating room may inadvertently touch the screw head and thus contaminate it, which can result in serious surgical damage.

With the known individual packaging, there was therefore an increased risk of contamination because the screw head was accessible from the upper insertion opening, which should be avoided.

With the known individual packaging, only a single elongated item can be packaged at a time, assuming that a screw head or a head with an enlarged diameter is present, because the holding function takes place in this region. It was therefore only a roughly linear, resiliently expandable clamping collar that carried out the holding function. The operation of this clamping collar was inconvenient due to a lack of sufficient gripping surfaces.

Furthermore, the well-known individual packaging depends on a specific head shape being specified, otherwise the packaging will not work.

The invention is therefore based on the object of developing a packaging for singular items of the type mentioned at the outset in such a way that the items to be packaged can be safely placed in a resiliently expandable packaging and that these items are protected against unintentional contact.

To achieve the object, the invention is characterized by the technical teaching of the independent claim.

It is preferred if one or more locking knobs are arranged in the transition region between the closure part and the packaging space, which locking knobs form a resiliently expandable closure gap therebetween and that a guide device is arranged in the packaging space in vertical alignment with the closure gap for guiding the lower part of the packaged item in a position-secured manner.

By arranging a guide device on the packaging space side to guide the lower part of the item to be held without tilting, the advantage is achieved that the lower part of the item is held in a sliding guide with radial play and cannot tilt in the packaging space.

It is preferable if the guide device consists of two vertical, parallel boundary ribs that are spaced apart from one another and form an approximately sleeve-shaped guide channel therebetween, in which the lower part of the item to be held is held with radial play in a position-secured manner and in the manner of a sliding guide.

The profile shape of the guide channel is preferably adapted to the shape of the item to be held in its lower region. A round profiled guide channel is therefore suggested for a round profiled item. However, if the lower part of the item is polygonal or asymmetrical, for example, it is preferable if the guide channel is also adapted to this polygonal or asymmetrical profile, so that the item to be held is held in the guide channel with sliding clearance and secured against twisting. This means that the item can only be removed from the individual packaging in a single twisted position.

In order to prevent the guide device from being resiliently deformed, it is preferred if the mutually parallel vertical boundary ribs are rigid, which is achieved by the fact that the boundary ribs preferably connect the front wall to the rear wall of the packaging body over their entire length and are thus designed to be relatively rigid and resistant to bending.

Overall, it is a flat packaging bag in whose packaging space a singular item is stored in a position-secured manner and free of tilt, and which can be removed by finger pressure on the gripping surfaces via the closure gap that then opens.

Removal can be achieved by turning the individual packaging upside down and then opening the closure gap by pressing the gripping surfaces by finger pressure, after which one or more items fall out due to gravity.

In another variant, the item can be removed from the packaging space using a magnetic tool or a suitable gripping tool when the packaging is upright and the closure gap is open.

The arrangement of locking knobs, which limit the packaging space at the top and form the closure gap, has the advantage that in the event of incorrect operation, for example if the medical assistant holds the packaging upside down and wants to empty it, the items stored in the packaging space are prevented from accidentally falling out. The locking knobs, which can only be activated by finger pressure and form the closure gap, are a safety device against accidental emptying.

It is preferred if there is a resiliently expandable, flat packaging body overall, the upper closure part of which is resiliently expandable, wherein the closure part is formed by at least two gripping ribs arranged opposite one another on the narrow sides of the packaging body, which can be resiliently pressed against one another, whereby the closure gap arranged in the lower region of the closure part and formed by one or more locking knobs widens.

The widened closure gap allows the item held in a position-secured manner in the packaging space to be safely removed from the packaging when it is facing downwards with the removal opening.

Alternatively, it can be removed with the packaging upright using a suitable tool.

It is preferred if the one or more locking knobs are arranged on the inner side of the front wall and the rear wall of the closure part, facing one another and aligned with one another, whereby they move away from one another when suitable deformation pressure is applied to the opposing gripping surfaces and thereby form an open closure gap through which the item can be inserted or removed.

According to the invention, it is therefore preferred if the upper closure part of the packaging body is delimited by an opening that can be widened by finger force, and that beyond this opening one or more opposing locking knobs are arranged in alignment on the front and rear walls, which form an expandable closure gap therebetween.

The advantage of the given technical teaching is that the item to be packaged no longer necessarily has to be a screw with a screw head with an enlarged diameter, but can be any shaped item that passes through the opening of the packaging, widened in the upper region, and through the widened closure gap underneath into the lower packaging space of the packaging body, without there being a risk of contact with the item in the upper region of the opening of the packaging body.

This means that improved sterile requirements can be met and a singular item can be securely packaged because it is held in the packaging region without coming into contact with it when the closure gap is closed.

This is where the invention differs from the prior art according to DE10 2013 019 452 B4, because the shape of the packaging had to be adapted to the shape of the item to be held. With the invention, this is no longer necessary. This also eliminates the need to provide a large number of packaging variants for a large number of different singular items to be packaged, because different singular items can be packaged in a single package. Such an item can be a pin, a screw, a nut, a plate or other small-part items found in the medical field.

When the opening of the packaging is widened, any singular item can pass through the expandable closure gap arranged at a distance below the mouth of the packaging and can no longer leave the packaging when the closure gap is closed.

The locking force for the locking knobs forming the closure gap is defined by the resilient restoring force of the plastics material in the neck region of the packaging.

In another embodiment of the invention, it can additionally be provided that the locking force (closing force) of the closure gap is enlarged by the locking knobs forming a toothed engagement.

Because the technical teaching of the invention now makes it possible for the first time to form a protected packaging space in the individual packaging in which an item can be stored in a position-secured manner, there is the further advantage that in the upper region, i.e. beyond the locking knobs and therefore in the region of the upper edge, it is possible for the first time to securely close the entire opening by means of a closure means.

A first secure closure of the opening edge is, for example, that the plastics material from which the packaging body is made can be welded by means of welding in order to create an absolutely tight packaging, which was not possible with the known individual packaging according to the prior art.

Another closure option is, for example, that the opening of the packaging body in the upper region of the peripheral edge can be closed with a sealed and tear-off closure sheet or with a closure adhesive.

In any case, it is important in the invention that a protected packaging space is formed beyond and below the locking knobs and that the region of the packaging body located above the locking knobs can be sealed in any desired manner, with an airtight seal, for example by welding, being particularly preferred.

Since it is a packaging for sterilizable items, such packaging bodies are preferably used in the medical field and the items to be packaged are preferably medical, sterilizable items, such as screws, knobs, nuts and pins, and the like. Sterilization is preferably carried out on the packaging filled with packaging items and tightly sealed by means of radiation sterilization.

In another preferred embodiment, it is provided that the resiliently expandable individual packaging with contents is placed in an airtight bag packaging and then sterilized.

The packaging body should be easy to handle. It should preferably be operated with the fingers of one hand. Therefore, the preferred dimensions of the packaging are in the range of a length of, for example, 40 mm up to a length of 400 mm. A preferred width is about 26 mm up to a preferred width of about 200 mm.

These are merely preferred dimensions which do not limit the scope of the invention.

A further characteristic of the packaging according to the invention is that it is a resiliently deformable flat body made of plastics material, preferably transparent- or also slightly colored—i.e., a body which consists of two walls parallel to one another, namely a rear wall and a front wall, and the two walls are parallel to one another and are laterally connected by narrower side walls, whereby an overall approximately flat, i.e., pocket-shaped, body with an approximately rectangular cross section is formed.

A preferred shape of the packaging body is a rectangular shape or a shape approximating to a rectangular shape with rounded corners.

Another design provides that the parallel front and rear walls have a slightly bulbous (convex) shape. The side walls can also have a convex shape.

The rectangular shape has the advantage that the packaging is protected against accidentally rolling away from a storage surface.

The device is operated by finger pressure on opposing gripping ribs, which are arranged in the region of the upper closure part. The actuation force corresponds approximately to the finger force required to open a clothespin.

The approximately flat, oval cross-sectional shape of the packaging body is converted into an approximately round shape in the neck region. As a result, the previously oval opening in the upper region of the closure part widens and the locking knobs arranged in the lower region of the closure part, which are aligned with one another and form the closure gap therebetween, move away from one another, whereby the closure gap opens and an item can be removed or inserted from the lower packaging space through the opened closure gap.

For larger packaging bodies, it is provided that locking knobs are arranged in pairs on both the front and rear walls, each opposite one another. This means that the locking knobs are arranged in pairs on the front wall and in pairs on the rear wall and are aligned in a horizontal line to one another and form the closure gap therebetween that is expandable by finger pressure or—in the case of very large packaging—by machine pressure.

The packaging body is preferably designed to be resilient in its upper neck region so that the gripping ribs can be pressed against one another by finger pressure in order to widen the peripheral opening edge. This results in a large operating surface that can also be easily operated with a gloved hand.

The invention is not limited to this. In another embodiment, in the case of larger packaging, it may be provided that machine assistance is also used for resilient deformation of the neck region for filling or removing packaging items.

However, for smaller packaging bodies it is sufficient to arrange only one pair of opposing locking knobs in the lower part of the neck region. Therefore, only one locking knob is provided on each side of the packaging. In the case of larger packaging, a plurality of locking knobs can be arranged at a distance from one another on each side of the package, which are in gap engagement with the locking knobs arranged on the opposite side of the package to form the closure gap.

The shape of the locking knobs can be varied within wide limits. It can be provided that the locking knobs safely limit the closure gap in the closed position, but on the other hand do not prevent the packaged item from being shaken out or slipping out. Therefore, the locking knobs in the gap engagement can be spaced apart from one another. In this variant, there is therefore no frictional mutual contact between the locking knobs.

In another variant, the locking knobs in the gap engagement can have a mutual frictional contact.

It is also preferred if the locking knobs arranged in pairs form a mutual distance between themselves, because when such a packaging body is compressed, the material between the locking knobs bends in the region of this intermediate distance and thus an improved widening of the opening and the closure gap is possible.

In another embodiment of the invention, it can be provided that the locking knobs arranged in pairs on the front wall and the rear wall are connected to one another, i.e., the central distance between the locking knobs-which previously formed the kink region—is then eliminated. This ensures that the two locking knobs arranged in pairs merge into a single horizontal elongated locking knob on the front wall and the rear wall, whereby it is always assumed that the locking knobs on the front and rear walls are aligned with one another.

Furthermore, the invention does not depend on the locking knobs being arranged on both the front wall and the rear wall. In a different embodiment, it can be provided that either only on the inner side of the front wall or only on the inner side of the rear wall there are individual locking knobs or locking knobs arranged in pairs, which form a closure gap to the smooth inner side of the opposite wall, whereby the closure gap is then no longer arranged centrally in the packaging body, but is offset laterally.

In a further embodiment of the invention, it is provided that the locking knobs arranged opposite one another on the front wall and the rear wall are not aligned with one another, but are laterally offset from one another on a horizontal line so that they form an interlocking row of teeth.

The locking knobs arranged at a distance on the front wall then engage in the gaps between the locking knobs arranged on the rear wall in the manner of a toothed engagement and thus form a toothed, labyrinth-like closure gap, which therefore has a special closure characteristic. In a first embodiment, the toothed engagement can allow mutual play between the interlocking locking knobs. In a second embodiment, the toothed engagement can also be frictional, i.e. the interlocking “teeth” of the locking knobs form an additional locking force, the amount of which exceeds the resilient restoring force of the plastics material in the neck region.

The locking knobs, which are directed against one another in pairs and form a mutual toothed engagement, do not only have to be present in pairs, but in another variant of the invention it is sufficient that one locking knob is arranged on the front side and an opposite locking knob is arranged on the rear side and the two locking knobs are not aligned with one another but are offset laterally from one another in order to also allow for a toothed engagement and thus form a bent closure gap.

Thus, while the first embodiments, which are preferably described in the drawings, describe a horizontal, straight continuous closure gap, in the variants just described the locking knobs are arranged in such a way that they form a mutual toothed engagement so as not to form an elongated, flat closure gap, but a serrated, interlocking closure gap.

In all embodiments, the lower part of the item to be held can be made to stand up against stop knobs on the packaging side to further improve the positional stability, in order to avoid limiting the displacement position of the item in the packaging body. Such stop knobs can preferably form flat stop surfaces for the lower end face of the item. In another embodiment, they can also be designed with insertion bevels.

The subject matter of the present invention results not only from the subject matter of the individual claims, but also from the combination of the individual claims with one another.

All information and features disclosed in the documents, including the abstract, in particular the spatial configuration shown in the drawings, could be claimed as substantial to the invention insofar as they are new compared to the prior art, individually or in combination. The use of the terms “substantial” or “according to the invention” or “substantial to the invention” is subjective and does not imply that the features so named must necessarily be part of one or more claims.

In the following, the invention is explained in more detail with reference to drawings which merely illustrate one embodiment. Further substantial features and advantages of the invention will become apparent from the drawings and their description.

### BRIEF DESCRIPTION OF THE DRAWINGS

In the drawings:

FIG. 1 is a view of a first embodiment of a packaging body;

FIG. 1a is the top view of the opening of the packaging body according to FIG. 1;

FIG. 2 is a longitudinal section through the packaging body along the section line A-A in FIG. 1;

FIG. 3 is a view of a second version of a packaging body;

FIG. 4 is a section through the packaging body according to section line B-B in FIG. 3;

FIG. 4a is a top view of the second packaging body in the direction of its opening;

FIG. 5 is a view of a third version of a packaging body Side view of the packaging body according to FIGS. 1 and 2;

FIG. 6 is a top view of the third packaging body in the direction of its opening;

FIG. 7 is a view of the first packaging body according to FIG. 1 in the widened state;

FIG. 8 is the top view of the deformed opening of the packaging body shown in FIG. 7;

FIG. 9 is a view of the second packaging body according to FIG. 3 in the widened state;

FIG. 10 is the top view of the deformed opening of the packaging body shown in FIG. 9;

FIG. 11 is a view of the third packaging body according to FIG. 3 in the widened state;

FIG. 12 is the top view of the deformed opening of the packaging body shown in FIG. 11;

FIG. 13 is a view of a first variant of the third packaging body according to FIG. 5 in an undeformed state;

FIG. 14 is a top view of the third packaging body according to FIG. 13 in the direction of its opening;

FIG. 15 is a view of a second variant of the third packaging body according to FIG. 5 in an undeformed state;

FIG. 16 is a top view of the third packaging body according to FIG. 15 in the direction of its opening;

FIG. 17 is a view of a third variant of the third packaging body according to FIG. 5 in an undeformed state;

FIG. 18 is a top view of the third packaging body according to FIG. 17 in the direction of its opening;

FIG. 19 is a view of the third packaging body according to FIG. 13 in a deformed state;

FIG. 20 is a top view of the packaging body according to FIG. 19 in the direction of its opening in a deformed state;

FIG. 21 is a view of the packaging body according to FIG. 15 in a deformed state;

FIG. 22 is a top view of the packaging body according to FIG. 21 in the direction of its opening in a deformed state;

FIG. 23 is a view of the packaging body according to FIG. 17 in a deformed state;

FIG. 24 is a top view of the packaging body according to FIG. 23 in the direction of its opening in a deformed state;

FIG. 25 is a view of a fourth variant of the packaging body according to FIG. 9 in an undeformed state;

FIG. 26 is a top view of the packaging body according to FIG. 25 in the direction of its opening in an undeformed state;

FIG. 27 is a view of a fourth variant of the packaging body according to FIG. 25 in a deformed state;

FIG. 28 is a top view of the packaging body according to FIG. 27 in the direction of its opening in a deformed state;

FIG. 29 is a top view of the packaging body according to FIG. 25-28 with a packaged item;

FIG. 30 is a top view of the opening of the packaging body according to FIG. 29;

FIG. 31 is a section through the packaging body according to section line B-B in FIG. 30;

FIG. 32-34 is a step-by-step representation of the loading or unloading of the packaging body with an item;

FIG. 35 is a section through the packaging body according to FIG. 1 or 7 in the deformed state when removing an item;

FIG. 36 is the top view of the removal opening;

FIG. 37 is the side view of the packaging body according to FIG. 35;

FIG. 38-40 is a step-by-step representation of the removal of the item from the packaging body according to FIG. 35-37;

FIG. 41 is a section through a loaded packaging body according to FIGS. 15 and 21 with another packaging item;

FIG. 42 is a plan view of the mouth of the packaging body according to FIGS. 41 and 43;

FIG. 44-46 is a step-by-step representation of the removal of the item from the packaging body according to FIG. 41-43;

### DETAILED DESCRIPTION

In all the drawings, the packaging body 1 according to the invention has a substantially flat, elongated body shape, i.e., it is approximately rectangular in cross section, because it comprises a flat front wall 2, a rear wall 4 parallel thereto, and two side walls 3 adjoining in one piece.

The entire packaging body 1 is preferably manufactured in one piece using the blow molding process and consists of a resilient plastics material, preferably a TPU plastics material. Therefore, it is sterilizable, weldable, and abrasion-resistant. It is preferably transparent or lightly colored and permanently resilient. It forms a flat packaging bag. It preferably has a hardness in the range of 45 to 95 shore-A and particularly preferably a hardness of 85 shore-A+−5.

The preferred dimensions have already been mentioned in the general description part, whereby the packaging body according to the invention is intended to be suitable for the packaging of relatively small items, as they occur in the medical field, whereby the term “small items” also includes organ replacement parts, such as artificial knee joints, shoulder joints, bone nails, hip joints, bone saw blades and/or small parts, such as screws, nuts, pins, plates and the like. The type and arrangement of the means arranged in the interior of the packaging for securing the position and guiding the packaged item, as well as the lower stop knobs limiting the position, are adapted to the body of the item to be packaged. For this reason, only a few examples of the packaging of different items are given in the following drawings.

The embodiments shown in the drawings show preferred actual dimensions of a packaging body in a 1:1 scale for the sterile packaging of individual screws, nuts, pins, plates, and the like.

The packaging space 27 receiving the items is elongated and closed off at the bottom by a rounded bottom part 5. However, the packaging space 27 and the base part 5 can only be resiliently compressed to a small extent, because the compressibility is limited by the guide device 22 arranged in the packaging space 27.

This resilient deformability of the neck region 8 facilitates the removal of items 17 stored in the packaging space 27.

The transition from the packaging space 27, which is oval in cross section, to the oval neck region 8 of a closure part 6, which adjoins the top, takes place via an arched convex contour. The neck region 8 is therefore reduced in width in cross section compared to the packaging space 27. It forms an upper opening 9, which can be converted by resilient deformation into an opening 9′ that approximates a round shape.

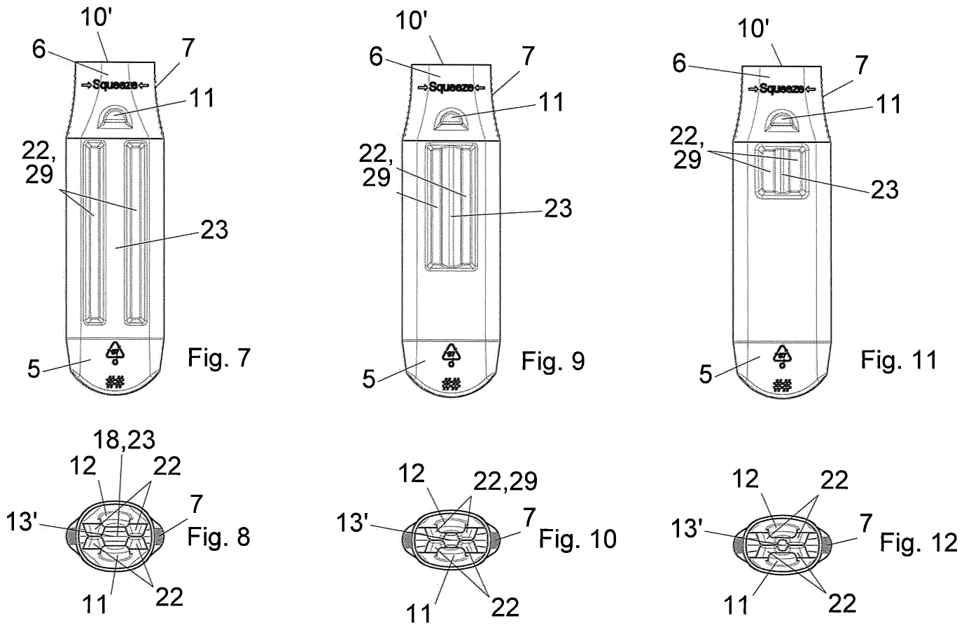

The undeformed oval opening 9 is defined by a peripheral edge 10 and in the region of the side walls 3, in the region of the closure part 6, opposite gripping ribs 7 are arranged in alignment with one another, which can be squeezed against one another by finger pressure or—in the case of automatic packaging machines—with the aid of a machine. The resulting shape is described in the following FIG. 7 et. seq.

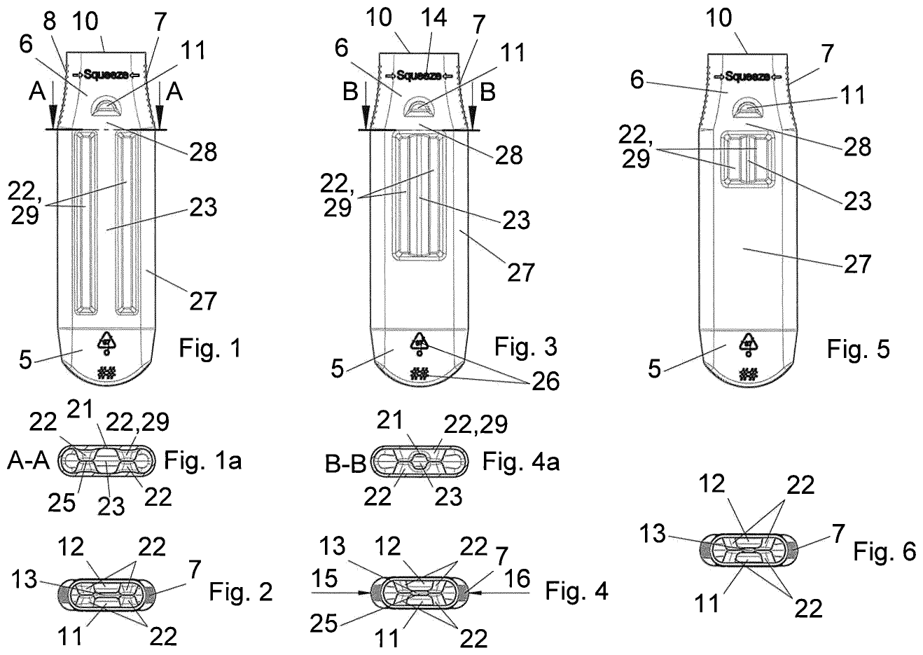

It is important that a locking mechanism is arranged in the lower region of the closure part 6 and in the transition to the packaging space 27, which in the embodiment shown is formed from pairs of opposing locking knobs 11, 12 that are arranged in alignment with one another, with a pair of locking knobs 11 being arranged on the inner side of the front wall 2 and an identical pair of locking knobs 12 being arranged on the inner side of the rear wall 4, and the pairs of locking knobs 11, 12 being arranged in alignment with one another, as can be seen from FIG. 1a, 2, 4, 4a, 6. In the closed state, they therefore form a closure gap 13 running in a horizontal direction, which in the closed state can have a preferred width in the range of 0 mm to 1 mm.

In the general description it has already been pointed out that it is not necessary for the solution that the locking knobs 11, 12 arranged in pairs opposite one another touch one another. However, in another embodiment it can be provided that the locking knobs 11, 12 are offset from one another in order to form a toothed, sealed, or unsealed engagement with one another.

It has also already been mentioned in the general description that instead of the arrangement of paired locking knobs 11, 12, individual locking knobs 11, 12 can also be provided.

However, more than two locking knobs can be arranged on one inner side of the front wall or one inner side of the rear wall.

Accordingly, the number of locking knobs arranged on the front wall 2 and on the rear wall 4 is not limited. There may be more than two.

It is preferred if a label 14 is arranged in the region of the closure part 6, which indicates that the gripping ribs 7 can be compressed against one another in the directions of arrows 15, 16 (see FIG. 4) by finger pressure in order to bring the packaging into an open state.

FIG. 2-6 show further details of the construction, whereby it can also be seen from FIG. 2 that there is a distance between the locking knobs 11, 12 arranged in pairs, which distance acts as a kink region 21. The arrangement of the kink region 21 allows for the material to bulge out convexly in the region of the kink region 21 when the gripping ribs 7 are compressed against one another, in order to allow for a reduction in the compression force when the closure part 6 is opened. This reduces the compression force caused by finger pressure and at the same time increases the width of the closure gap 15. In all cases, regardless of whether a kink region 21 is present or not, the compression force preferably corresponds to the force that must be applied to open a clothespin.

In another embodiment, it can be provided that the locking knobs 11, 12 arranged in pairs on the front and rear walls are each formed into a single horizontal locking knob.

When describing the present packaging body, it is irrelevant what shape the packaging items have, as shown below. This means that the packaging items can have a length such that they just touch the underside of the locking knobs 11, 12 in the packaging space 27 at the top and protrude into the bottom part 5 at the bottom. However, they can also be much shorter, so that the packaging has the characteristics of a pocket-shaped individual packaging.

A large number of individual items 17 can thus be fed into the packaging space 25 via the opened closure gap 13′ and then removed again when the closure gap 13′ is opened.

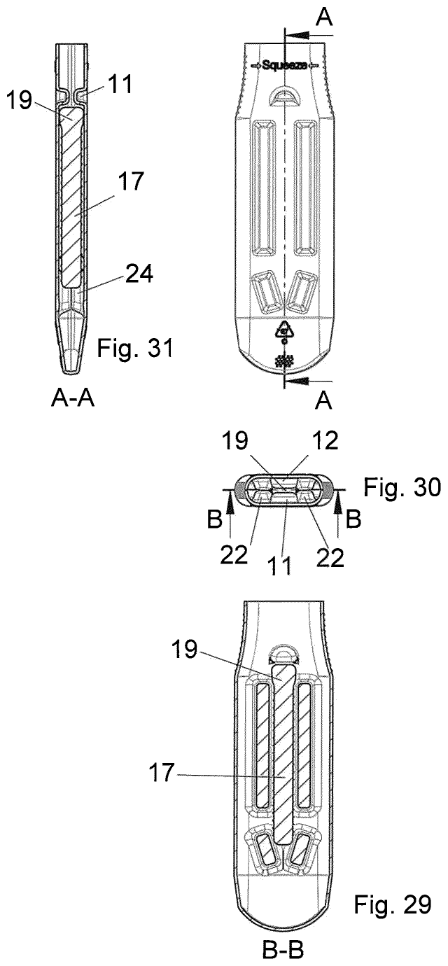

FIGS. 7 to 12 show the opened packaging body 1, assuming that pressure is exerted on the gripping ribs 7 in the directions of arrows 15, 16 with finger pressure or by machine and the opening 9 is transformed into an approximately round-profile- or enlarged oval-opening 9′. The closure gap 13 also opens into the closure gap 13′ because the locking knobs 11, 12 move away from one another and form the cross-sectionally enlarged and open closure gap 13′. As shown in FIGS. 29 to 46, the items 17 to be packaged can then be introduced into the packaging space 27 through the open closure gap 13′.

FIG. 1a and 2 show that the locking knobs 11, 12 are profiled in a specific way, i.e., they preferably have lateral insertion slopes, so that an item to be packaged can be inserted particularly easily via the insertion bevels.

Such insertion bevels or exit bevels arranged at the bottom can also be omitted.

A comparison of FIGS. 1, 3 and 5 shows that the guide device 22 arranged in the interior can have a different axial length for the same package size. The length of the guide device 22 depends on the kind of item to be held and on the properties that are to be assigned to this guiding device.

Each guide device 22 consists of 2 lateral, groove-shaped boundary ribs 29, which are each notched parallel to one another at a mutual distance in the front and rear walls 2, 4 and are connected to one another in their mutual contact area in the packaging space 27 via the connecting region 25. They therefore connect the front wall 2 with the rear wall 4. Accordingly, these are stable, flexurally rigid boundary ribs 29, which determine the length of the guide device 22 and which form therebetween an approximately round-profiled guide channel 23, which is suitable for receiving the lower part of the item 17. In this way, the item 17 to be packaged is held in a special position securing device in the guide channel 23 in the region of the guide device 22 on the packaging chamber side so that it cannot tilt.

As mentioned in the general description section, this guide channel 23 can also be profiled in a specific way in order to adapt to the profile of the item 17 to be held in this region. If such a profile is given, for example an oval, rectangular, or polygonal profile shape, the item to be held is secured against twisting in the guide channel 23 and can no longer rotate around its own longitudinal axis.

The guide channel 23 is preferably arranged in the longitudinal center region of the guide device 22.

The rigid connection of the axial notches of the guide device ensures that it is not bent or deformed in any other way, even if the packaging body 1 is squeezed.

FIGS. 2, 4, 4a and 6 each show the view of the undeformed opening 9 with the peripheral edge 10. They are the same for the packaging bodies 1 shown in FIGS. 1, 3 and 5.

FIG. 7, 9, 11 show the same packaging bodies 1 according to FIGS. 1, 3 and 5, but with the neck region 8 in a deformed state.

From the representations in FIGS. 8, 10 and 12 it can be seen that only the closure gap 13 has been converted into the open closure soon 13 line and therefore the locking knobs 11, 12 have moved away from one another and as a result the guide channel 23 is open and is no longer closed by the locking knobs 11, 12.

The locking knobs 11, 12 thus form a passage channel 18 therebetween, which is axially aligned with the guide channel 23 of the guide device.

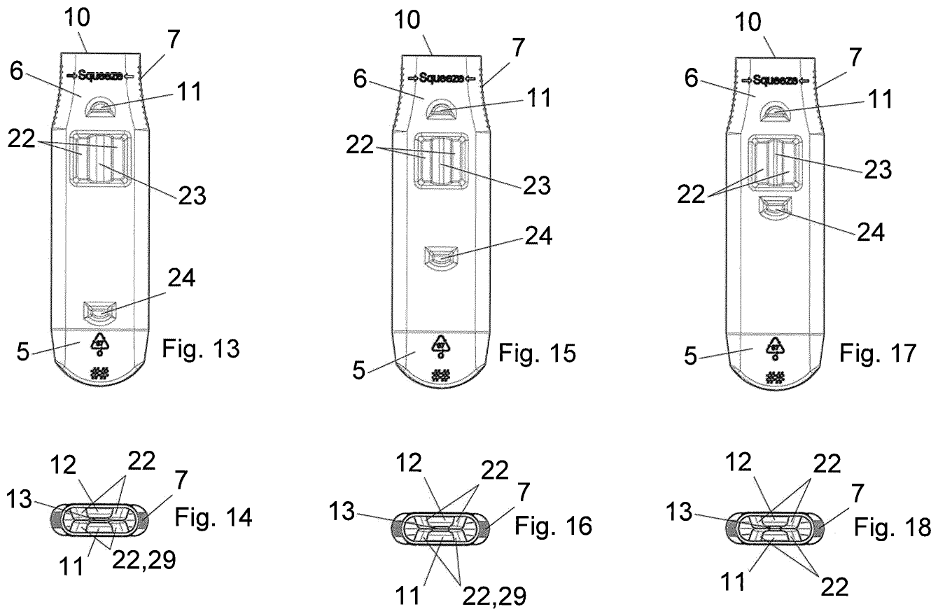

FIGS. 13, 15 and 17 show further embodiments of a packaging body, which differ from the previously described embodiments only in that one or more stop knobs 24 are arranged in the region on the underside of the packaging space 27, which are provided to stop the lower end face of the item to be held.

The stop knobs 24 can form a flat contact surface for the item resting thereon. In other embodiments, however, they can also be adapted to the profile shape of the item and thus form an anti-rotation lock or have one or more insertion bevels.

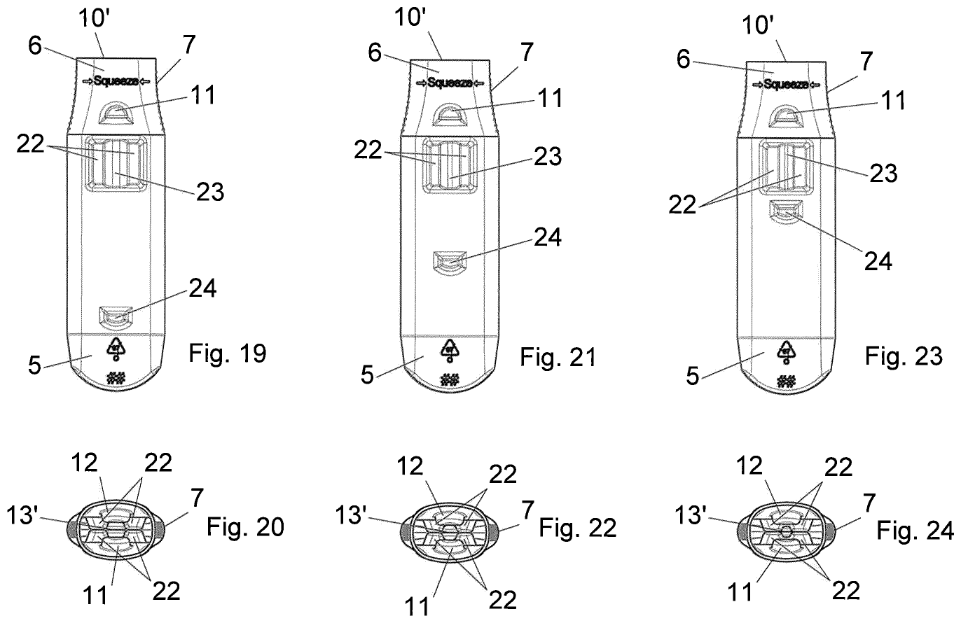

FIG. 19, 21, 23 show the resiliently deformed packaging body according to FIGS. 13, 15, and 17. FIGS. 20, 22 and 24 therefore show the opening pattern of the packaging body with a representation of the resiliently widened opening 9′.

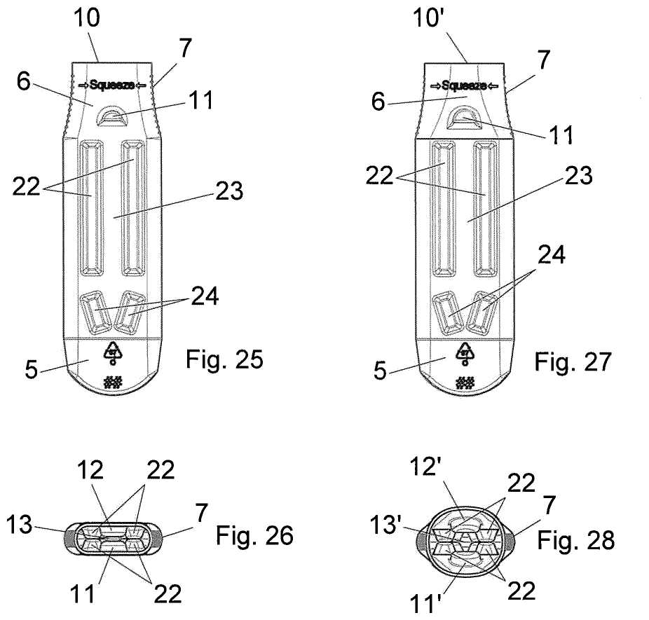

The embodiment example according to FIGS. 25 to 28 shows a further variant of a packaging body which differs from the preceding embodiments only in that in the packaging space 27 2 stop knobs 24 are arranged at a distance from one another, which are directed obliquely towards one another and thus form an upwardly open insertion bevel for an item to be held there. This results in even better centering of the item 17 to be held, which then takes place not only in the guide channel 23, but also in the region of the bottom-side stop knobs 24. FIGS. 26 and 28 show the view of the undeformed and the deformed opening 9.

FIGS. 29 to 34 show a preferred example of how an item 17 to be packaged is held in the packaging space 27 of a package shown in FIG. 25-28, with reference to the embodiment shown in FIG. 25-28. FIG. 31 shows that the screw head 19 of the item 17 rests on the underside of the locking knobs 11, 12 in a free space 28 and rests with its bottom end on the previously described stop knobs 24. This can also be seen in FIGS. 29 and 30. It is therefore a position-secured and vibration-protected holder for an item 17, the front, lower end of which engages in the stop bevels of the stop knobs 24 and is held there to prevent displacement. Such a holder has the advantage that the item 17 can no longer move in the packaging space 27, even under the effects of vibration forces, and does not inadvertently scrape off plastics material on its contact surfaces on the packaging side and thus become contaminated.

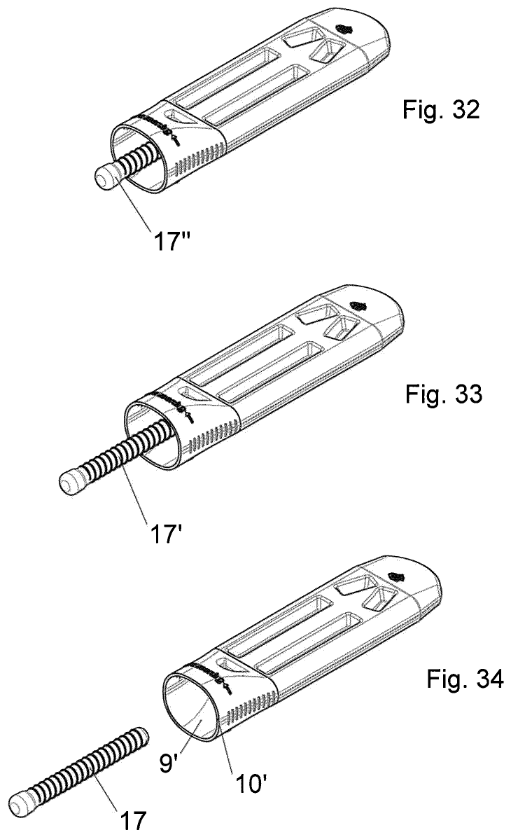

FIG. 32-34 show the step-by-step insertion or removal of an item 17, 17′ and 17″ from a package according to FIG. 25-31. The item 17 in the form of a screw can also be removed by pouring when the opening 9′ is squeezed together.

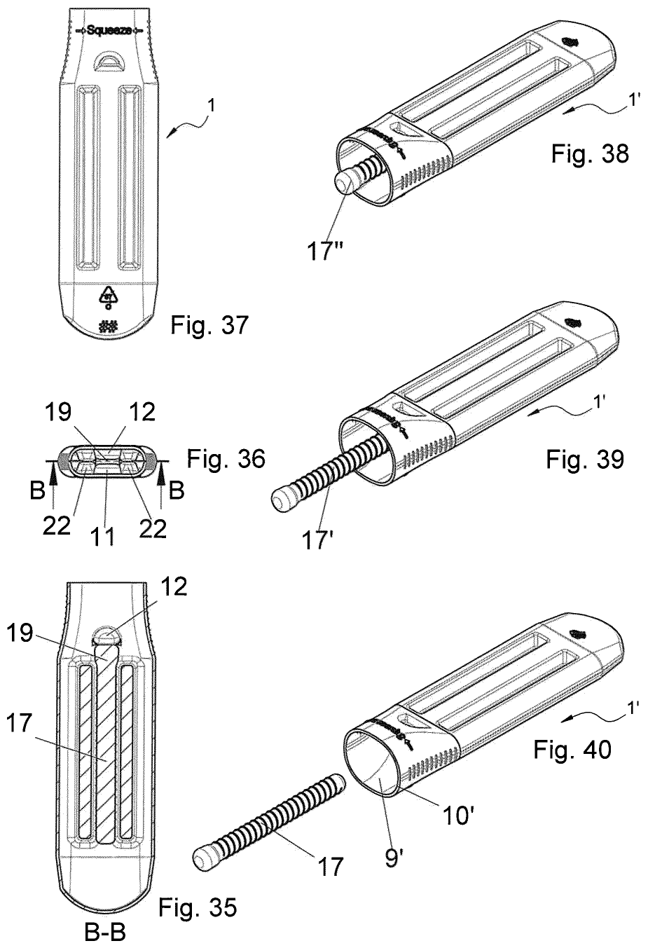

FIGS. 35 to 40 show the same process as described in FIG. 32-34 with a packaging body 1 according to one of the embodiments shown in FIGS. 1 and 7. In contrast to the embodiment shown in FIG. 29-31, the screw head 19 is secured against displacement with its upper side on the underside of the locking knobs 11, 12 and with its underside on the inlet region of the guide channel 23. A bottom stop on the base side is not required.

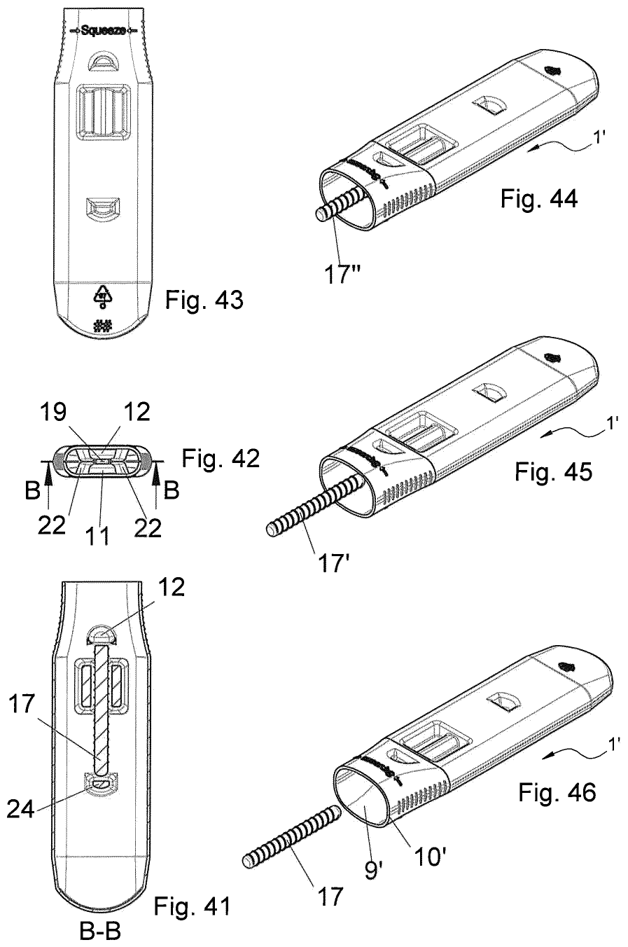

FIGS. 41 to 44 show a modification of the embodiment according to FIGS. 35 to 40 with the difference that the lower end of the item rests on a lower stop knob 24 and instead of the screw there is a pin which has no screw head 19.

### LIST OF REFERENCE SIGNS

1) Packaging body
1′) Packaging body
2) Front wall
3) Side wall
4) Rear wall
5) Base part
6) Closure part
7) Gripping rib
8) Neck region (of 6)
8′) Neck region (of 6)
9) Opening, 9′
10) Edge
10′) Edge
11) Locking knob (front)
12) Locking knob (rear)
13) Closure gap 13′
14) Labeling
15) Arrow direction
16) Arrow direction
17) Item, 17′, 17″
18) Passage channel
19) Screw head
20)
20′)
21) Kink region
22) Guide device
23) Guide channel (with 29)
24) Stop knob
25) Connecting region (of 22)
26) Labeling symbol
27) Packaging space
28) Free space
29) Boundary rib (for 23)

1. An elastically expandable, sterilizable individual packaging for elongated items, the packaging comprising:
a packaging body open on one side, which has a packaging space closed at a bottom and merges into a resiliently expandable closure part on its upper side, herein one or more locking knobs are arranged in the transition region between the closure part and the packaging space, which locking knobs form a resiliently expandable closure gap therebetween and in that a guide device is arranged in the packaging space in vertical alignment with the closure gap for guiding the lower part of the packaged item in a position-secured manner.

2. The packaging according to claim 1, wherein
the packaging body is designed as a resiliently expandable, flat, pocket-shaped plastics material body.

3. The Individual packaging according to either claim 1 wherein the closure part is resiliently expandable by finger force.

4. The Individual packaging according to claim 1, wherein at least two gripping ribs are arranged on the closure part and are arranged opposite one another on the narrow sides of the packaging body, which are resiliently deformable in a direction directed towards one another.

5. The Individual packaging according to claim 4, wherein the locking knobs for forming the closure gap are arranged in the space between the at least two opposing gripping ribs.

6. The Individual packaging according to claim 1, wherein a packaging space is designed to hold a single item, which is held by a guide device, and in that there is a free space in an axial distance between the guide channel and the locking knobs.

7. The Individual packaging according to claim 6, wherein at least one bottom-side stop knob is arranged in the packaging space in an aligned extension of the guide channel of the guide device, on which stop knob the lower end of the item to be held rests.

8. The Individual packaging according to claim 1, where the locking knobs are arranged individually or in pairs on the inner sides of a front wall and a rear wall and in horizontal alignment with one another and form the resiliently expandable closure gap.

9. The packaging according to claim 6, wherein the guide device consists of two boundary ribs which are parallel to one another and form a mutual spacing from one another and which form therebetween a sleeve-like guide channel for longitudinal guidance of a part of the item in the packaging space.

10. The packaging according to claim 9, wherein in that the boundary ribs connect a front wall and a rear wall of the packaging body to one another over their entire length.

11. The packaging according to claim 1, wherein charge that the transition from an oval packaging space into an oval neck region of the closure part adjoining at the top is effected via an arched convex contour, so that the oval neck region is reduced in width in cross section compared to the oval packaging space.

12. The packaging according to claim 11, wherein the neck region forms an upper opening which is defined by a peripheral edge and that the opening is resiliently expandable.

13. The packaging according to claim 11, wherein the oval opening of the neck region can be closed by welding or gluing or sealing.

14. The packaging according to claim 1, characterized in that the packaging body is manufactured in one piece using the blow molding process and consists of a resilient plastics material, preferably a TPU plastics material.

15. The packaging according to claim 1, characterized in that it consists of a plastics material with a hardness in the range of 45 to 95 shore-A and particularly preferably has a hardness of 85 shore-A+−5.

16. An elastically expandable, sterilizable individual packaging for elongated items, the packaging comprising:
a packaging body having an open first side, the packaging body forming a packaging space closed at a bottom side,
the packaging space having a resiliently expandable closure on an upper side, wherein one or more locking knobs are arranged in a transition region between the resiliently expandable closure and the packaging space, the locking knobs forming a resiliently expandable closure gap therebetween, and
wherein a guide device is arranged in the packaging space in vertical alignment with the resiliently expandable closure for guiding a lower part of the packaged elongated item in a position-secured manner.
